(19) United States
(12) Patent Application Publication
(10) Pub. No.: US 2007/0298995 A1
(43) Pub. Date: Dec. 27, 2007

(54) Fragrance-containing compositions comprising deliquescent substances

(75) Inventors: Marcus Eggers, Beverungen (DE); Sabine Widder, Holzminden (DE); Manfred Meier, Furstenberg (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/763,683

(22) Filed: Jun. 15, 2007

(30) Foreign Application Priority Data
Jun. 21, 2006 (EP) 06 115 795.4
Jul. 13, 2006 (EP) 06 117 145.0

(51) Int. Cl. A61K 8/34

(52) U.S. Cl. 512/2; 512/25; 512/3

(57) ABSTRACT

The present invention relates to a composition comprising or consisting of: A) from 45 to 99 wt. % ethanol, based on the total weight of the composition, B) one or more fragrances, C) water, D) (i) one or more deliquescent substances.

### FIELD OF THE INVENTION

The present invention relates to specific ethanolic fragrance compositions (fragrance mixtures), to the use of deliquescent substances for achieving specific effects in ethanolic fragrance compositions, to processes for the preparation of ethanolic fragrance compositions according to the invention, and to methods for achieving specific effects in ethanolic fragrance compositions, wherein deliquescent substances are used, and to methods of conveying, enhancing or modifying an odour on human skin. A particular aspect of the present invention relates to improving the adhesion of ethanolic fragrance compositions to skin and/or hair.

### BACKGROUND OF THE INVENTION

In the fragrance sector, in particular in fine perfumery, it is generally known that, when ethanolic fragrance compositions (in particular perfumes, eau de toilette (EdT), etc.) are applied to the skin, evaporation of the ethanol and/or of the water that is generally present results in the loss of the readily volatile top note in particular, while the lower-volatility notes exhibit excellent adhesion to the skin and are released from the skin, and accordingly perceived in terms of their odour, over a long period of time. Because of the more rapid evaporation of the top note, there is consequently a marked change over time in the odour profile of ethanolic fragrance compositions applied to skin. A similar effect occurs when ethanolic fragrance compositions are applied to (human) hair. It should be noted in this connection that the described disadvantageous effect is experienced as a problem especially when an ethanolic fragrance composition (fragrance mixture) is used as a leave-on product, that is to say is to remain on the skin and/or hair and be released therefrom over a prolonged period of time. The loss of readily volatile top notes in particular is naturally less problematic where a (an ethanolic) fragrance composition is used only to achieve a short-term olfactory effect.

Within the scope of the present text, adhesion, adhesiveness and substantivity are understood as being the adhesion of a fragrance to skin and hair, in particular adhesion to human skin (with the exception of the (oral) mucosa). Therefore, although the present invention also concerns the (oral) mucosa in respect of the application of ethanolic fragrance compositions, it is to be noted that the significance of the present invention in terms of the aspects skin (with the exception of the (oral) mucosa) and/or hair (in particular human skin) is of far greater relevance, because when an ethanolic fragrance composition is applied to the (oral) mucosa, the negative evaporation effect is generally eclipsed and dominated by wash-away effects (supported in the region of the oral mucosa, for example, by the influence of saliva).

In perfumistic practice, many attempts have already been made to extend the adhesiveness or perceptibility in particular of the top note of ethanolic fragrance compositions in particular on human skin and accordingly achieve a certain olfactory “profile stability”.

For example, the use of so-called fixatives is described many times, that is to say the use of individual substances or combinations of (fragrance) substances that are added to more readily volatile fragrances or fragrance mixtures in order to reduce the evaporation rate thereof (see U.S. Pat. No. 6,737,396).

In IP.COM #000033581D (published January 2005), the use of hydroxyalkylurea derivatives, in particular hydroxyethylurea, is described, whereby the adhesive properties of perfumes in cosmetic applications, in particular aqueous-ethanolic formulations, to skin and hair is extended.

U.S. Pat. No. 3,939,099 describes the use of film formers which dissolve in a water/ethanol mixture and are miscible with fragrance (mixtures). Examples which are mentioned include ionic and non-ionic derivatives of water-soluble polymers, such as, for example, polyvinylpyrrolidone derivatives, quaternary polyvinyl-pyrrolidones having molar weights in the range from 50,000 to 1,000,000, cationic cellulose derivatives and the like. During the evaporation of ethanol a film forms on the skin, in which film the fragrances are embedded.

U.S. Pat. No. 6,210,688 describes the formation and use of an odourless polymer film on the skin (based on vinyl ether copolymers, polyacrylates, methacrylates, polyesters, polyfluorocarbons, polysaccharides), to which a perfume is subsequently applied. “Reactions” with the skin are said to be suppressed thereby.

FR 2 747 306 describes the use of polymeric hydrocarbons (polyethylenes having molecular weights from 3000 to 30,000). Because such polymers are insoluble in ethanol/water, a corresponding product, such as, for example, eau de cologne, EdT or an eau de parfum, is cloudy.

WO 2004/098556 describes a novel sprayable and clear perfume formulation that is distinguished by increased surface tension or a reduced contact area after application, which is achieved by the use of an effective amount of a polymer. Owing to the smaller contact area, the perfume oil that remains after evaporation of the ethanol is concentrated in a smaller area, from which the fragrances evaporate more slowly.

Film formers and/or polymers have the disadvantage that they reduce the evaporation rate not only of the top note but also of all the other, lower-volatility fragrances, resulting in a marked reduction in the overall intensity. Furthermore, film formers can cause an unpleasant feeling of stickiness or tightness on the skin.

EP 0 181 401 and EP 0 857 481 describe gel-like perfume preparations in which the diffusion, and hence also the evaporation, of readily volatile fragrances in particular is reduced. However, the proposed gel formation is not suitable in particular for the fine perfumery (fine fragrance) sector, because such preparations are not sprayable. In addition, the gel-like formulation affects not only the evaporation of the readily volatile fragrances (top note), but also the evaporation of lower-volatility base notes, which is noticeable overall in a reduction in the odour intensity (impact).

### SUMMARY OF THE INVENTION

The primary object of the present invention was, therefore, to provide an (alternative) ethanolic fragrance composition in which the fragrances used, but in particular readily volatile fragrances and in particular fragrances which form the top note of a scent, possess long (extended as compared with conventional fragrance compositions) adhesion in particular to (human) skin (which means skin excluding mucosa). The above-described disadvantages of fragrance compositions known hitherto are thereby to be avoided as far as possible.

The ethanolic fragrance composition to be provided is preferably to comprise one or more additives which influence the adhesion of the (in particular readily volatile) fragrances in the desired manner but otherwise have no effect or only an extremely small and therefore tolerable effect on the sensory properties of the ethanolic fragrance composition.

The ethanolic fragrance compositions to be provided are preferably to be transparent (clear) and/or sprayable.

The primary object is achieved according to the invention by a composition comprising or consisting of:
A) from 45 to 99 wt. % ethanol, based on the total weight of the composition,
B) one or more fragrances,
C) water,
D) (i) one or more deliquescent substances. In a composition according to the invention, the proportion of the sum of constituents A), B), C) and D) in the composition is preferably 95 wt. % or more, more preferably 98 wt. % or more.

The compositions according to the invention are distinguished in particular by
extended adhesion to skin and/or hair, in particular to human skin (which here means also human skin that is not (oral) mucosa), in particular of the top note;
an odour impression that remains largely constant over a prolonged period after application to human skin (see in this connection the examples below);
a greater impact after application to human skin.

### BRIEF DESCRIPTION OF THE DRAWINGS

The following is a brief description of the drawings, in which:

FIGS. 1 to 8 are graphs showing the absolute amount of a fragrance as a function of time.

### DETAILED DESCRIPTION OF THE INVENTION

Within the scope of the present text, a “deliquescent substance” is understood as being a substance which, in the pure, solid state, liquefies in air with sufficient atmospheric moisture.

Preference is given to a composition according to the invention in which in constituent D)
(i) the one or more deliquescent substances are one or more salts.

Preference is accordingly given to the use of deliquescent salts. Deliquescent salts liquefy in the free, solid state in air with sufficient atmospheric moisture by absorption of atmospheric moisture.

In a composition according to the invention, the amounts of constituent A) (ethanol) and constituent D) are preferably so chosen that constituent D) is dissolved completely in the composition without cloudiness.

Preference is given to compositions according to the invention in which constituent D)
(i) comprises one or more deliquescent salts selected from a group G, wherein the group G is a group consisting of: ammonium hydrogen carbonate, ammonium tricitrate, ammonium fluoride, ammonium formate, ammonium magnesium chloride, ammonium propionate, ammonium sulfamate, ammonium thiocyanate, barium iodide, barium bromide, barium chloride, barium thiocyanate, barium nitrate, caesium iodide, caesium chloride, caesium hydroxide, caesium carbonate, caesium fluoride, calcium bromide, calcium chloride, calcium iodide, calcium nitrate, calcium nitrite, calcium thiocyanate, cobalt diacetate, cobalt dibromide, cobalt dichlorate, cobalt diperchlorate, cobalt diiodide, copper dibromide, copper dichlorate, copper diperchlorate, copper fluorosilicate, copper dinitrate, lithium chlorate, lithium chloride, lithium bromide, lithium salicylate, lithium sulfide, magnesium acetate, magnesium bromide, magnesium chlorate, magnesium perchlorate, magnesium chloride, magnesium iodide, magnesium nitrate, manganese dichloride, potassium iodide, potassium formate, potassium hydroxide, silver perchlorate, sodium perchlorate, sodium hydroxide, sodium phenolate, sodium hypophosphite, sodium hydrogen sulfide, sodium sulfide, sodium thiocarbonate, sodium thiocyanate, titanium tetrabromide, titanium trichloride, zinc perchlorate, zinc chloride and zinc thiocyanate.

Preferred deliquescent salts (from group G) are:

ammonium hydrogen carbonate, ammonium tricitrate, ammonium fluoride, ammonium formate, ammonium magnesium chloride, ammonium propionate, ammonium sulfamate, ammonium thiocyanate, caesium iodide, caesium chloride, caesium hydroxide, caesium carbonate, caesium fluoride, calcium bromide, calcium chloride, calcium iodide, calcium nitrate, calcium thiocyanate, copper dinitrate, lithium chlorate, lithium chloride, lithium bromide, lithium salicylate, lithium sulfide, magnesium acetate, magnesium bromide, magnesium chlorate, magnesium perchlorate, magnesium chloride, magnesium iodide, magnesium nitrate, manganese dichloride, potassium iodide, potassium formate, sodium perchlorate, sodium phenolate, sodium hypophosphite, sodium thiocyanate, zinc perchlorate, zinc chloride and zinc thiocyanate.

Particularly preferred deliquescent salts (from group G) are:

ammonium hydrogen carbonate, ammonium fluoride, ammonium magnesium chloride, ammonium propionate, ammonium thiocyanate, caesium chloride, caesium fluoride, calcium bromide, calcium chloride, calcium iodide, calcium nitrate, copper dinitrate, lithium chloride, lithium bromide, magnesium acetate, magnesium bromide, magnesium chloride, magnesium iodide, magnesium nitrate, potassium nitrate and zinc chloride.

Most preferred deliquescent salts (from group G) are:

calcium chloride, calcium nitrate, copper dinitrate, lithium chloride and magnesium chloride, in particular calcium chloride and calcium nitrate.

Furthermore, the above preferred groups G are also in particular further preferred when the constituent lithium chloride in each case is not included in the respective preferred group.

Furthermore, zinc phenolsulfonate is not regarded as a deliquescent salt within the scope of the present invention.

In a process according to the invention for the preparation of a composition according to the invention (in particular in one of the forms described above as being preferred),
A) from 45 to 99 wt. % ethanol,
B) one or more fragrances (in particular readily volatile fragrances),
C) water,
D) (i) one or more deliquescent substances and/or (ii) two or more compounds that together form a deliquescent substance in situ in the resulting compound, and optionally further constituents are mixed. Percentages by weight are here again based on the total weight of the resulting composition after mixing.

Within the scope of the present text, the “two or more compounds that together form a deliquescent substance in situ in the resulting compound” are compounds that together form a deliquescent salt during or after incorporation into a composition according to the invention.

According to alternative (ii), therefore, it is possible to use, for example, compounds that form a deliquescent substance in situ, that is to say, for example, ammonium chloride and calcium propionate, which, after mixing with the constituents A), B) and C) (and optionally with further constituents, see below, in particular constituents E) and F)), are present in solution, whereby the solution can then be interpreted as formally comprising calcium chloride as well as ammonium propionate.

Constituent D) is preferably present in a composition according to the invention in completely dissolved form, in particular when a deliquescent salt is used or is formed in situ.

According to our own findings, the effectiveness of constituent D) of a composition according to the invention (i.e. in particular the effectiveness of a deliquescent salt) depends on how deliquescent the deliquescent substance(s) used are in the pure, solid state. According to our own investigations, the more pronounced the deliquescence of constituent D), the more pronounced the described effects in an ethanolic fragrance composition according to the invention. In particular, increasingly improved adhesion (in particular of the top note) to skin and/or hair, in particular to human skin, was observed as the deliquescence increased.

A proportional relationship between the amount of deliquescent substances (in particular deliquescent salts) used and the strength of the described effects has also been observed. In particular, when the amount of deliquescent salt added was doubled, the effect in a composition according to the invention was also seen almost to double, in particular improved adhesion (in particular of the top note) to skin and/or hair, in particular to human skin (i.e. not (oral) mucosa).

In a composition according to the invention (in particular in one of the forms described above as being preferred), constituent D) is preferably present in an effective amount, that is to say in an amount at which constituent D) brings about in the composition one or more effects selected from the group of effects consisting of:
a reduction in the evaporation of fragrances, in particular immediately after application of the composition according to the invention and in particular of readily volatile fragrances (in particular the top note); this is true especially of (readily volatile) fragrances that are usually carried away by the evaporation of ethanol/water shortly after application of an aqueous-ethanolic composition. No appreciable reduction in the evaporation of lower-volatility fragrances is associated with this effect; this is true especially of fragrances that exhibit more or less constant evaporation even in the absence of a constituent D) in an aqueous-ethanolic composition;
a stabilisation of the odour profile over time, that is to say an at least substantially constant odour profile is obtained over a prolonged period;
an increase in or stabilisation over time of the olfactory impact (the perceived strength of the odour); this means that the olfactory impact (the perceived strength of the odour) is not or not appreciably reduced over a long period of time; depending on the concentrations of constituent D) used in each particular case in the composition according to the invention, the impact is increased over a more or less long period of time;
an extension of the adhesion of the composition, in particular of the readily volatile fragrances (top note), to skin and/or hair, in particular to human skin;
an increase in or extension of the diffusivity (spatial effect) of the composition, in particular of the readily volatile fragrances (top note), on skin and/or hair, in particular on human skin;
the conveying of a pleasant skin sensation.

A composition according to the invention preferably does not leave behind a film on skin and/or hair and is accordingly not experienced as annoying. Shortly after application (that is to say after drying), a composition according to the invention is generally no longer visible (exception: hair care agents such as, for example, hairspray, in which film formation is desirable).

In summary, compositions according to the invention are accordingly compositions having particularly good adhesion of the top note of a fragrance mixture and an extended and/or enhanced impact of the top note, a lessening of the heart or base note not being observed, or at least not being observed to an appreciable extent.

Preference is given to a composition according to the invention, preferably in one of the forms described above as being preferred, comprising:
A) from 50 to 98.5 wt. %, preferably from 60 to 95 wt. %, ethanol, and/or
B) from 0.1 to 35 wt. %, preferably from 0.5 to 25 wt. %, of a fragrance mixture comprising 2, 3, 4, 5, 6, 7, 8, 9, 10 or more fragrances, and/or
C) from 0.5 to 40 wt. %, preferably from 0.5 to 25 wt. %, water, and/or
D) from 0.5 to 20 wt. %, preferably from 2 to 12 wt. %, more preferably from 3 to 8 wt. %, of one or more deliquescent salts.

Percentages by weight are in each case based on the total weight of the composition. Preferably, the preferred percentages by weight mentioned for constituents A), B), C) and D) are adjusted simultaneously.

A composition according to the invention (in particular in one of the forms characterised above as being preferred) can comprise as an additional constituent:
E) optionally glycerol, ethylene glycol, 1,2-propylene glycol, diethyl phthalate and/or a straight-chained alkanediol having from 6 to 12 carbon atoms.

A particularly preferred composition according to the invention comprises or consists of:
A) from 60 to 95 wt. % ethanol,
B) from 1 to 25 wt. % of a fragrance mixture comprising 2, 3, 4, 5, 6, 7, 8, 9, 10 or more fragrances,
C) from 1 to 22 wt. % water,
D) from 2 to 12 wt. %, preferably from 3 to 8 wt. %, of one or more deliquescent salts (preferably one or more of the salts characterised above as being preferred), and
E) optionally glycerol, ethylene glycol, 1,2-propylene glycol, diethyl phthalate and/or a straight-chained alkanediol having from 6 to 12 carbon atoms. The percentages by weight are in each case based on the total weight of the composition.

The optional constituent(s) E) glycerol, ethylene glycol, 1,2-propylene glycol, diethyl phthalate and/or straight-chained alkanediol having from 6 to 12 carbon atoms (in particular 1,2-pentanediol, 1,2-hexanediol and 1,2-octanediol and mixtures thereof) can be present, in particular in an aftershave as an example of a composition according to the invention, in an amount of up to 6 wt. %. The optional constituent(s) E) glycerol, ethylene glycol, 1,2-propylene glycol, 1,2-pentanediol, 1,2-hexanediol and/or 1,2-octanediol can act as solubilisers, so that any undesirable cloudiness and/or separation is suppressed.

A composition according to the invention preferably has one, two or all of the properties from the group consisting of: transparent (clear), true solution, capable of being sprayed (sprayable). A composition according to the invention is therefore preferably transparent (clear) and/or a true solution and/or capable of being sprayed (sprayable).

Constituent B) of a composition according to the invention (in particular in one of the forms characterised above as being preferred) preferably comprises one or more fragrances from the group consisting of:

alpha-pinene, beta-pinene, limonene, 6-methyl-5-hepten-2-one, octanal, eucalyptol (1,8-cineol), rose oxide, 3-hexenol, dihydromyrcenol (2,6-dimethyl-7-octen-2-ol), menthone, isomenthone, 2,6-dimethyl-5-hepten-1-al, 3-hexenylmethylcarbonate, benzaldehyde, linalool, linalyl acetate, caryophyllene, ethyl linalool, citral, neral, geranial, benzyl alcohol, p-anisaldehyde, menthol, hexyl acetate, citronellol, nerol, geraniol and 2-phenylethyl alcohol. When the mentioned fragrances are used, the above-mentioned effects are particularly pronounced.

The mentioned preferred fragrances of constituent B) can, where appropriate, be in the form of their respective diastereoisomers, enantiomers and/or double bond isomers. For example, they can be in the form of (E)/(Z) isomers, in the form of any desired mixture of the enantiomers, in particular in the form of the racemate, or alternatively in the form of any desired mixture of the corresponding diastereoisomers.

A preferred composition according to the invention comprises as constituent B) a fragrance mixture containing 2, 3, 4, 5, 6, 7, 8, 9, 10 or more fragrances, comprising or consisting of
i) a top note, containing or consisting of one or more fragrances each having a vapour pressure at 25° C. greater than or equal to 1.333 Pa.

Preferably, therefore, constituent B) of a composition according to the invention comprises at least one fragrance that is particularly readily volatile (vapour pressure at 25° C. greater than or equal to 1.333 Pa) and that forms the top note or is a constituent of the top note. Within the scope of the present text, a readily volatile fragrance is generally understood as being a sensorially active substance having a vapour pressure greater than or equal to 1.333 Pa (which corresponds to about 0.01 torr) at 25° C., it frequently being advantageous, however, to use readily volatile fragrances having a vapour pressure greater than or equal to 2.000 Pa (which corresponds to about 0.015 torr), in particular to use fragrances having a vapour pressure greater than or equal to 2.666 Pa (which corresponds to about 0.02 torr), in each case at 25° C., as the top note or as a constituent of the top note.

When the fragrances, or mixtures thereof, mentioned hereinbelow were used, the above-mentioned effects were particularly pronounced, and these fragrances are therefore most preferred within the scope of the present invention:

alpha-pinene, D-limonene, 6-methyl-5-hepten-2-one, octanal, eucalyptol (1,8-cineol), cis-rose oxide, trans-rose oxide, dihydromyrcenol (2,6-dimethyl-7-octen-2-ol), d-menthone, 1-menthone, 2,6-dimethyl-5-hepten-1-al, (Z)-3-hexenylmethyl-carbonate, linalool, linalyl acetate, citral, neral, geranial, I-menthol, racemic menthol, hexyl acetate, citronellol and 2-phenylethyl alcohol.

Preference is given to a composition according to the invention in which constituent B) is a fragrance mixture containing 2, 3, 4, 5, 6, 7, 8, 9, 10 or more fragrances, comprising or consisting of:
i) a top note containing or consisting of one or more fragrances each having a vapour pressure at 25° C. greater than or equal to 1.333 Pa, and
ii) one or more fragrances each having a vapour pressure at 25° C. less than 1.333 Pa.

In a particularly preferred composition according to the invention, lower-volatility fragrances are therefore present in addition to a top note.

Preference is given to a composition according to the invention in which constituent B) is a fragrance mixture containing 2, 3, 4, 5, 6, 7, 8, 9, 10 or more fragrances, comprising or consisting of:
i) a top note containing or consisting of 2, 3, 4, 5, 6 or more fragrances each having a vapour pressure at 25° C. greater than or equal to 1.333 Pa, and
ii) 2, 3, 4, 5, 6 or more fragrances each having a vapour pressure at 25° C. less than 1.333 Pa.

In addition to constituents A), B), C), D) and optionally E), a composition according to the invention can additionally comprise as an additional constituent, that is to say as a constituent that cannot be subsumed under the above definitions relating to constituents A), B), C), D) and E), one or more substances from the group consisting of:

F) antioxidants (such as, for example, BHT (p-tert.-butylhydroxytoluene), BHA (p-tert.-butylhydroxyanisole), tocopherol (vitamin E), tocopheryl acetate), skin-care substances (alpha-bisabolol, allantoin, aloe Vera, panthenol, camomile extract), citric acid, vitamin C, UV filters, emulsifiers, fatty alcohols, C₈-C₃₀-fatty acid alkyl esters, colourings.

In a preferred composition according to the invention, constituent F) is preferably present only in an amount of 2 wt. % or less, based on the total weight of the composition.

The compositions according to the invention can be used in many perfumed, in particular cosmetic products.

It should be noted that some of the fragrances preferably used in compositions according to the invention are used as sensorially active substances in conventional alcoholic oral care products (oral hygiene products), for example menthol, menthone, eucalyptol, linalool, alpha-pinene. However, because the primary object of the present invention was to improve the adhesion of the top note of a fragrance mixture to skin and/or hair, in particular under leave-on conditions, where the fragrance composition is not washed off the skin and/or hair, it will be understood that a composition according to the invention is preferably not an oral care product (oral hygiene product). Thus, the following salts, for example, are frequently used in conventional oral hygiene products and these salts are preferably not present in compositions according to the invention or are present in concentrations that are not conventional in oral hygiene products:

sodium monofluorophosphate, tin difluoride, zinc citrate, zinc sulfate, tin pyrophosphate, tin dichloride, potassium citrate, potassium nitrate, potassium chloride, strontium chloride, Cu salts, alkali fluorides such as Na fluoride, alkaline earth fluorides, ammonium fluoride.

Compositions according to the invention are particularly preferably even wholly free of tin salts and/or zinc salts and/or copper salts (with the exception of copper dinitrate) and/or alkali fluorides and/or alkaline earth fluorides. Particularly preferably, none of the mentioned salts is present in a composition according to the invention.

In addition, oral hygiene compositions frequently have a peppermint, spearmint or eucalyptus odour and/or taste owing to the presence of flavourings such as menthol, menthone and eucalyptol. Preferred compositions according to the invention do not have such an odour and/or taste.

Compositions according to the invention are preferably free of peppermint oil, spearmint oil and/or eucalyptus oil.

A composition according to the invention is preferably selected from the group consisting of (the preferred maximum ethanol content is given in brackets in each case): extract, eau de parfum, eau de toilette, eau de cologne (in each case up to 99 wt. %), splash cologne, aftershave, deodorant formulations, hair-setting lotions, hairspray, hair gels (in each case up to 50 wt. %), face lotion (up to 70 wt. %), hair lotion (up to 75 wt. %), hand disinfection agents (up to 99 wt. %), liquids and emulsions for freshening wipes (up to 90 wt. %).

The compositions according to the invention are preferably so-called “leave-on” products, that is to say products that remain on the skin and/or hair and are generally not washed off. They include especially eau de parfum, eau de toilette, eau de cologne and aftershaves.

In addition, compositions according to the invention can also be, for example, in one of the following (but less preferred) forms: deodorant spray, roll-on deodorant, hairspray. These are likewise “leave-on” products.

The present invention relates also to the use of a deliquescent substance (as defined above, preferably of a deliquescent substance described above as being particularly preferred) as an agent for
reducing the evaporation of fragrances in an ethanolic fragrance composition, in particular immediately after application and in particular of readily volatile fragrances;
stabilising the odour profile of an ethanolic fragrance composition over time;
increasing or stabilising over time the olfactory impact (the perceived strength of the odour) of an ethanolic fragrance composition;
extending the adhesion of fragrances of an ethanolic fragrance composition, in particular of the readily volatile fragrances (top note), to skin and/or hair, in particular to human skin;
increasing or extending the diffusivity (spatial effect) of an ethanolic fragrance composition, in particular of the readily volatile fragrances (top note), on skin and/or hair, in particular on human skin; and/or
conveying or enhancing a pleasant skin sensation of an ethanolic fragrance composition.

The present invention relates also to a method for
reducing the evaporation of fragrances in an ethanolic fragrance composition, in particular immediately after application and in particular of readily volatile fragrances;
stabilising the odour profile of an ethanolic fragrance composition over time;
increasing or stabilising over time the olfactory impact (the perceived strength of the odour) of an ethanolic fragrance composition;
extending the adhesion of fragrances of an ethanolic fragrance composition, in particular of the readily volatile fragrances (top note), to skin and/or hair, in particular to human skin;
increasing or extending the diffusivity (spatial effect) of an ethanolic fragrance composition, in particular of the readily volatile fragrances (top note), on skin and/or hair, in particular on human skin; and/or
conveying or enhancing a pleasant skin sensation of an ethanolic fragrance composition,

comprising the following step:
mixing
A) from 45 to 99 wt. % ethanol,
B) one or more fragrances,
C) water, and optionally further constituents with
D) (i) one or more deliquescent substances and/or (ii) two or more compounds that together form a deliquescent substance in situ in the resulting composition,

and optionally further constituents,

wherein the percentages by weight are based on the total weight of the resulting composition after mixing.

The compositions according to the invention can be prepared in a simple manner by mixing the individual components of constituents A), B), C) and D) as well as optionally E) and F), or of constituents A) to D) as well as optionally E) and F), or of the optionally pre-mixed constituents A), B), C) and D) as well as optionally E) and F). The order in which the individual components or constituents are brought into contact with one another is not critical and can be varied.

The present invention relates also to a method for
conveying, enhancing or modifying an odour on human skin,
extending the adhesion of fragrances of an ethanolic fragrance composition, in particular of the readily volatile fragrances (top note), to skin and/or or hair, in particular to human skin,
increasing or extending the diffusivity (spatial effect) of an ethanolic fragrance composition, in particular of the readily volatile fragrances (top note), on skin and/or hair, in particular on human skin and/or
conveying or enhancing a pleasant skin sensation of an ethanolic fragrance composition,

comprising the following step:
application of a composition according to the invention (preferably of a composition described above as being preferred) to human skin.

Parts and percentages in the Examples which follow are in each case by weight.

### EXAMPLES

### Example 1

### Top Note to be Used According to the Invention as Constituent B) to be Used

| | | Vapour pressure | |
|---|---|---|---|
| No. | Component | (torr) at 25° C. | Wt. % |
| 1 | Limonene | 2.330 | 10 |
| 2 | Hexyl acetate | 1.560 | 5 |
| 3 | Octanal | 1.170 | 5 |
| 4 | Methylheptenone | 2.700 | 5 |
| 5 | 2,6-Dimethyl-5-hepten-1-al | 0.759 | 5 |
| 6 | Rose oxide cis | 0.660 | 5 |
| 7 | Rose oxide trans | 0.660 | 5 |
| 8 | Cis-3-hexenol | 0.554 | 5 |
| 9 | Dihydromyrcenol | 0.124 | 5 |
| 10 | Menthone | 0.278 | 5 |
| 11 | (Z)-3-Hexenylmethylcarbonate | 0.187 | 5 |
| 12 | Linalool | 0.0577 | 10 |
| 13 | Linalyl acetate | 0.0826 | 5 |
| 14 | Menthol | 0.0232 | 5 |
| 15 | Citronellol | 0.013 | 8 |
| 16 | Citral | 0.149 | 2 |
| 17 | Phenylethyl alcohol | 0.0161 | 5 |
| 18 | Diethyl phthalate (solvent) | | 2.5 |
| 19 | Propylene glycol (solvent) | | 2.5 |
| | Total: | | 100.0 |

### Example 2

### Headspace Analysis of the Evaporation Rate of Individual Fragrances from the Skin

Test Compositions

| | P1 | Ref1 |
|---|---|---|
| Ingredients | wt. % | wt. % |
| CaCl₂ | 5 | — |
| Individual fragrance to be | 0.125 | 0.125 |
| tested (see below) | | |
| Ethanol | 75 | 78.9 |
| Water | 19.875 | 20.975 |
| Total: | 100 | 100 |

By means of a microlitre syringe, 50 μl of each perfume formulation P1 and Ref1 (not according to the invention) were applied to a defined area (2.5×2.5 cm) of the test subjects' forearm.

5 minutes after application, the evaporation rate of the individual fragrances was measured by means of the dynamic headspace method. A defined stream of air was passed through a bell jar attached to the forearm and the fragrances were concentrated by means of adsorption on sampling tubes containing activated carbon. Concentration was carried out for a total of 5 hours. The sampling tubes were exchanged after 10, 15, 25, 40 minutes, 1 hour, 2, 3, 4 and 5 hours and were analysed individually.

The concentrated fragrances are eluted with diethyl ether and quantified by means of GC/MS after addition of a standard.

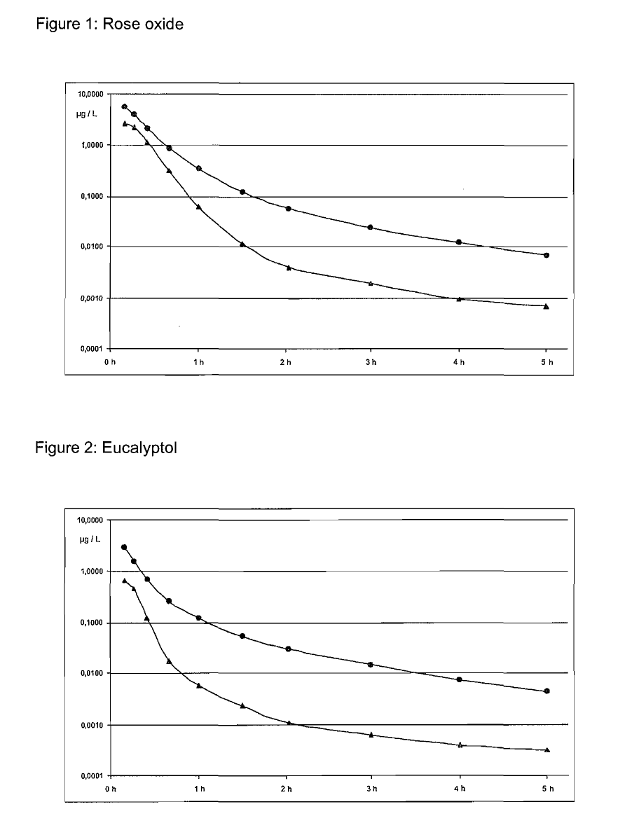

In FIGS. 1 to 8, the absolute amount of each fragrance tested (in μg per litre of air, y-axis) as a function of time (in hours, x-axis) is shown in graph form. It is to be noted in these figures that the y-axis represents a logarithmic scale. The results for the composition according to the invention are marked in each case by a circle, those for the reference samples by a triangle.

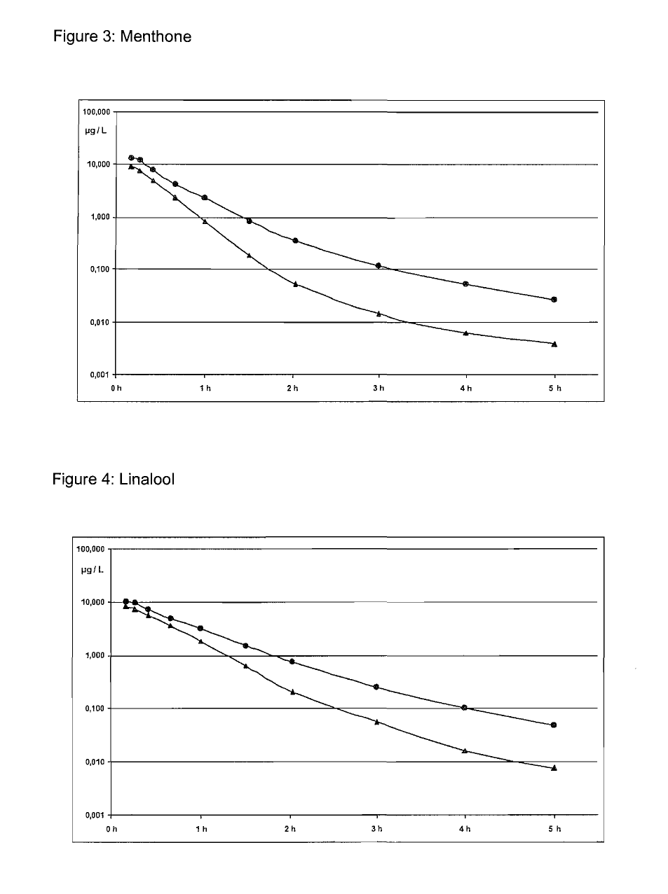

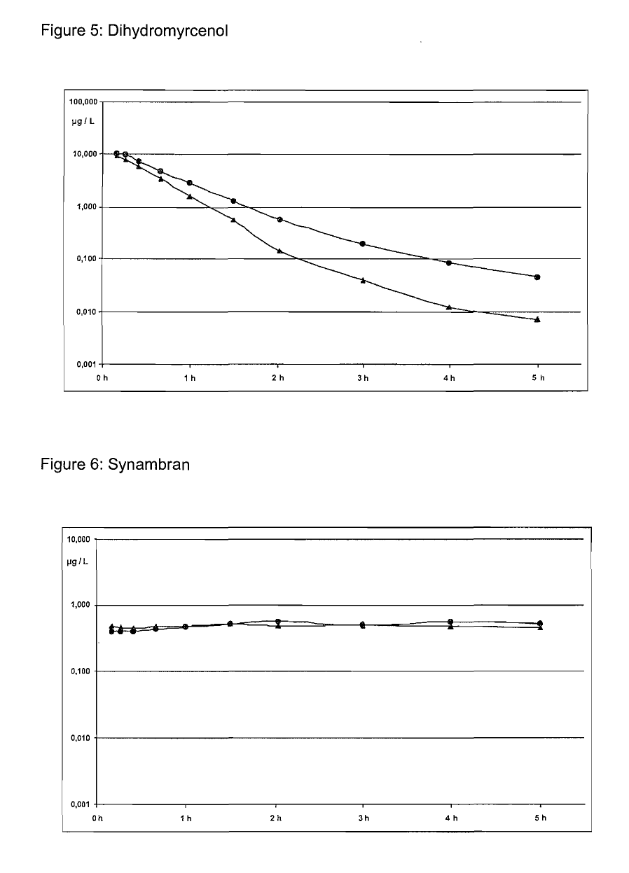

FIGS. 1 to 5 show by way of example the evaporation curve for the readily volatile fragrances rose oxide (FIG. 1), eucalyptol (FIG. 2), menthone (FIG. 3), linalool (FIG. 4) and dihydromyrcenol (FIG. 5) which are preferably to be used according to the invention, after application of the composition according to the invention (P1) and of the reference sample (Ref1).

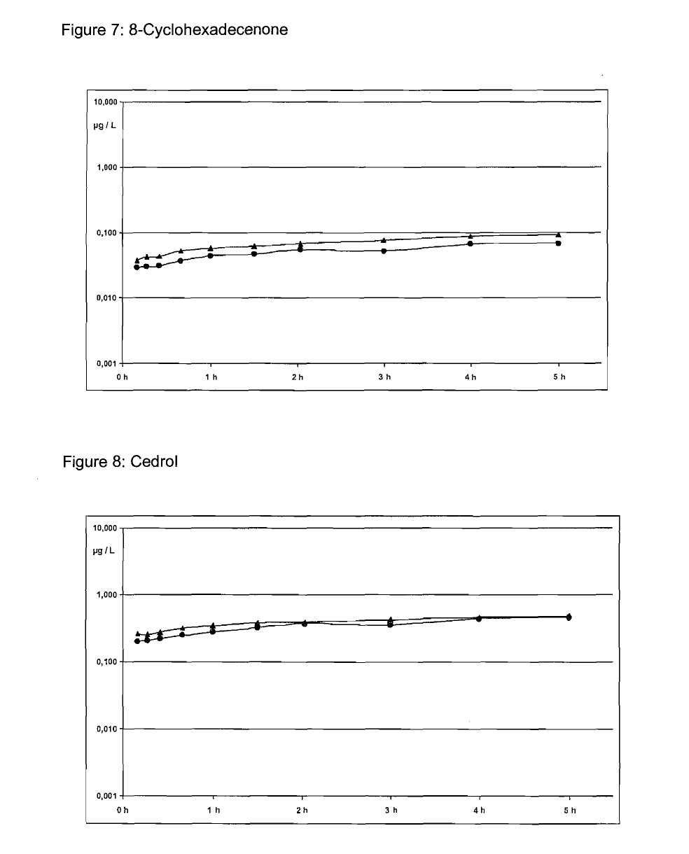

FIGS. 6 to 8 show by way of example the evaporation curve for the lower-volatility, that is to say non-volatile, fragrances Synambran (3a,6,6,9a-tetramethyldodecahydronaphtho[2,1-b]furan) (FIG. 6), 8-cyclohexadecenone (FIG. 7) and cedrol (FIG. 8).

For compositions that are preferred according to the invention (FIGS. 1 to 5), the results show markedly higher headspace concentrations for readily volatile fragrances (top note) over the entire period, while the lower-volatility fragrances (FIGS. 6 to 8), the evaporation rate of which is almost constant over the entire application cycle, remain largely unaffected.

In compositions according to the invention, therefore, the perceptibility of readily volatile fragrances in particular is extended and enhanced.

### Example 3

### Eau de Cologne

| | P2 | Ref2 |
|---|---|---|
| Ingredients | wt. % | wt. % |
| CaCl₂ | 5 | — |
| Fragrance mixture (top note) from Example 1 | 5 | 5 |
| Ethanol | 72 | 76 |
| Water | 18 | 19 |
| Total: | 100 | 100 |

Composition Ref2 is a comparison example and is not in accordance with the invention.

Sensory Evaluation of the Intensity

The intensity of the composition P2 according to the invention and of the corresponding reference Ref2 during an application cycle was assessed by a sensory panel. To this end, 2 sprays from a dispenser containing composition P2 according to the invention were applied to one forearm of a test subject, and 2 sprays of the reference Ref2 were applied to the other forearm.

The intensity of the perfume (composition) in each case was assessed by 16 trained panellists after 1 h, 2 h, 3 h and 4 h.

Table 1 shows the assessment of the 16 panellists in respect of the olfactory intensity of each composition.

TABLE 1

| Time | P2 stronger | No difference | Ref2 stronger |
|---|---|---|---|
| 1 h | 13/16 | 2/16 | 1/16 |
| 2 h | 14/16 | 2/16 | 0/16 |
| 3 h | 14/16 | 2/16 | 0/16 |
| 4 h | 13/16 | 3/16 | 0/16 |

### Example F1

### Deodorant Pump Spray

| | P1 |
|---|---|
| Ingredients | wt. % |
| CaCl₂ | 4.0 |
| Fragrance mixture from Example 1 | 0.8 |
| Perfume base B1 | 0.4 |
| Ethanol | 74.1 |
| Water | 19.0 |
| alpha-Tocopherol | 0.20 |
| Cocoamidopropyl PG-dimonium chloride phosphate | 0.40 |
| Chremophor RH 40 | 0.70 |
| Rosemary extract, soluble in ethanol | 0.20 |
| Myristyl alcohol | 0.20 |
| Total: | 100 |

Chremophor RH 40: polyoxyethylene glycerol tri-hydroxystearate 40, poly(oxyethylene)-40-hydrogenated castor oil (BASF AG)

The perfume base B1 consisted of:

11 wt. % benzyl acetate, 5 wt. % cyclohexyl acetate, 3 wt. % decanol, 4 wt. % amylcinnamaldehyde, 1 wt. % 3a,6,6,9a-tetramethyldodecahydronaphtho[2,1-b]-furan (Synambran (10% in DPG)), 9 wt. % rosewood oil, 4 wt. % allyl caproate, 10 wt. % geraniol, 4 wt. % 8-cyclohexadecenone, 4 wt. % eugenol, 10 wt. % methyl dihydrojasmonate (Hedion®), 12 wt. % jasmine absolute, 4 wt. % ylang ylang oil, 3 wt. % 2-methyl-3-(4-tert.-butylphenyl) propanal (Lilial®), 5 wt. % 2-methyl-5-phenylpentanol (Rosaphen), 4 wt. % 2-heptylcyclopentanone, 3 wt. % ethylene brassylate, 2 wt. % dihydrocoumarin, 1 wt. % vanillin.

### Example F2

### Aerosol Pump Spray

| | P1 |
|---|---|
| Ingredients | wt. % |
| CaCl₂ | 4.48 |
| Fragrance mixture from Example 1 | 1.1 |
| Perfume base B1 from Example F1 | 0.8 |
| Ethanol | 87.0 |
| Water | 5.0 |
| alpha-Tocopherol | 0.20 |
| Hydroxypropylcellulose | 0.20 |
| Rosemary extract, soluble in ethanol | 0.22 |
| Cetyl alcohol | 1.00 |
| Total: | 100 |

The indicated ingredients were homogenised, and then 60 parts by weight of the mixture were introduced into a spray can with 40 parts by weight of dimethyl ether (propellant).

### Example F3

### Non-Aerosol Hairspray with Extra-Strong Hold

| | I (wt. %) | II (wt. %) |
|---|---|---|
| Ethanol | 58.00 | 58.00 |
| Fragrance mixture from Example 1 | 0.30 | 0.30 |
| Calcium chloride (CaCl₂) | 2.00 | 2.00 |
| Zinc chloride (ZnCl₂) | 1.00 | — |
| Lithium chloride (LiCl) | 1.00 | 3.00 |
| Sulfonated polyesters (e.g. comb polymers | 8.00 | 10.00 |
| according to DE 199 09 757) | | |
| Vitamin E acetate and preservatives | 0.10 | 0.10 |
| UV filter | 0.10 | 0.10 |
| Distilled water | ad 100.00 | ad 100.00 |

1. A composition comprising:
A) from 45 to 99 wt. % ethanol, based on the total weight of the composition,
B) one or more fragrances,
C) water,
D) (i) one or more deliquescent substances.

2. A composition according to claim 1, wherein the proportion of the sum of constituents A), B), C) and D) in the composition is 95 wt. %.

3. A composition according to claim 1, wherein in constituent D) (i) the one or more deliquescent substances are one or more salts.

4. A composition according to claim 1, wherein constituent D) (i) comprises one or more deliquescent salts selected from a group G,
wherein the group G is a group of deliquescent salts consisting of: ammonium hydrogen carbonate, ammonium tricitrate, ammonium fluoride, ammonium formate, ammonium magnesium chloride, ammonium propionate, ammonium sulfamate, ammonium thiocyanate, barium iodide, barium bromide, barium chloride, barium thiocyanate, barium nitrate, caesium iodide, caesium chloride, caesium hydroxide, caesium carbonate, caesium fluoride, calcium bromide, calcium chloride, calcium iodide, calcium nitrate, calcium nitrite, calcium thiocyanate, cobalt diacetate, cobalt dibromide, cobalt dichlorate, cobalt diperchlorate, cobalt diiodide, copper dibromide, copper dichlorate, copper diperchlorate, copper fluorosilicate, copper dinitrate, lithium chlorate, lithium chloride, lithium bromide, lithium salicylate, lithium sulfide, magnesium acetate, magnesium bromide, magnesium chlorate, magnesium perchlorate, magnesium chloride, magnesium iodide, magnesium nitrate, manganese dichloride, potassium iodide, potassium formate, potassium hydroxide, silver perchlorate, sodium perchlorate, sodium hydroxide, sodium phenolate, sodium hypophosphite, sodium hydrogen sulfide, sodium sulfide, sodium thiocarbonate, sodium thiocyanate, titanium tetrabromide, titanium trichloride, zinc perchlorate, zinc chloride and zinc thiocyanate.

5. A composition according to claim 4, wherein the group G further comprises a group of deliquescent salts consisting of: calcium chloride, calcium nitrate, copper dinitrate, lithium chloride and magnesium chloride.

6. A composition according to claim 1, wherein constituent D) is dissolved completely in the composition.

7. A composition according to claim 1, wherein constituent D) is present in the composition in an effective amount, an effective amount being present when constituent D) brings about in the composition one or more effects selected from the group of effects consisting of:
a reduction in the evaporation of fragrances, in particular immediately after application and in particular of readily volatile fragrances;
a stabilisation of the odour profile over time;
an increase in or stabilisation over time of the olfactory impact (the perceived strength of the odour);
an extension of the adhesion of the composition, in particular of the readily volatile fragrances (top note), to skin (i.e. not (oral) mucosa) and/or hair;
an increase in or extension of the diffusivity (spatial effect) of the composition, in particular of the readily volatile fragrances (top note), on skin (i.e. not (oral) mucosa) and/or hair;
the conveying of a pleasant skin sensation.

8. A composition according to claim 1, further comprising:
A) from 50 to 98.5 wt. %, ethanol, and/or
B) from 0.1 to 35 wt. %, of a fragrance mixture comprising 2, 3, 4, 5, 6, 7, 8, 9, 10 or more fragrances, and/or
C) from 0.5 to 40 wt. %, water, and/or
D) from 0.5 to 20 wt. %, of one or more deliquescent salts, and
E) optionally glycerol, ethylene glycol, 1,2-propylene glycol, diethyl phthalate and/or a straight-chained alkanediol having from 6 to 12 carbon atoms,
wherein percentages by weight are based on the total weight of the composition.

9. A composition according to claim 1, further comprising:
A) from 60 to 95 wt. % ethanol,
B) from 1 to 25 wt. % of a fragrance mixture comprising 2, 3, 4, 5, 6, 7, 8, 9, 10 or more fragrances,
C) from 1 to 22 wt. % water,
D) from 2 to 12 wt. %, of one or more deliquescent salts, and
E) optionally glycerol, ethylene glycol, 1,2-propylene glycol, diethyl phthalate and/or a straight-chained alkanediol having from 6 to 12 carbon atoms,
wherein percentages by weight are based on the total weight of the composition.

10. A composition according to claim 1, wherein the composition has one, two or all of the properties selected from the group consisting of: transparent (clear), true solution, capable of being sprayed (sprayable).

11. A composition according to claim 1, wherein constituent B) of the composition further comprises one or more fragrances selected from the group consisting of:
alpha-pinene, beta-pinene, limonene, 6-methyl-5-hepten-2-one, octanal, eucalyptol (1,8-cineol), rose oxide, 3-hexenol, dihydromyrcenol (2,6-dimethyl-7-octen-2-ol), menthone, isomenthone, 2,6-dimethyl-5-hepten-1-al, 3-hexenylmethylcarbonate, benzaldehyde, linalool, linalyl acetate, caryophyllene, ethyl linalool, citral, neral, geranial, benzyl alcohol, p-anisaldehyde, menthol, hexyl acetate, citronellol, nerol, geraniol and 2-phenylethyl alcohol.

12. A composition according to claim 1, wherein constituent B) is a fragrance mixture containing 2, 3, 4, 5, 6, 7, 8, 9, 10 or more fragrances, comprising
i) a top note containing or one or more fragrances each having a vapour pressure at 25° C. greater than or equal to 1.333 Pa.

13. A composition according to claim 1, wherein constituent B) is a fragrance mixture containing 2, 3, 4, 5, 6, 7, 8, 9, 10 or more fragrances, further comprising
i) a top note containing one or more fragrances each having a vapour pressure at 25° C. greater than or equal to 1.333 Pa, and
ii) one or more fragrances each having a vapour pressure at 25° C. less than 1.333 Pa.

14. A composition according to a claim 1, wherein constituent B) is a fragrance mixture containing 2, 3, 4, 5, 6, 7, 8, 9, 10 or more fragrances, further comprising
i) a top note containing or consisting of 2, 3, 4, 5, 6 or more fragrances each having a vapour pressure at 25° C. greater than or equal to 1.333 Pa, and
ii) 2, 3, 4, 5, 6 or more fragrances each having a vapour pressure at 25° C. less than 1.333 Pa.

15. A composition according to claim 1, additionally comprising
F) one or more substances selected from the group consisting of: antioxidants, BHA, tocopherol, skin-care substances, citric acid, vitamin C, UV filters, emulsifiers, fatty alcohols, C₈-C₃₀-fatty acid alkyl esters and colourings.

16. A composition according to claim 15, wherein constituent F) is present in an amount of 2 wt. % or less, based on the total weight of the composition.

17. A composition according to claim 1, wherein the composition is selected from the group consisting of: extract, eau de parfum, eau de toilette, eau de cologne, splash cologne, aftershave, deodorant formulations, hair-setting lotions, hairspray, hair gels, face lotions, hair lotions, hand disinfection agents, liquids and emulsions for freshening wipes.

18. A composition according to claim 1, wherein a deliquescent substance is added as an agent for
reducing the evaporation of fragrances in an ethanolic fragrance composition, in particular immediately after application and in particular of readily volatile fragrances; or
stabilising the odour profile of an ethanolic fragrance composition over time; or
increasing or stabilising over time the olfactory impact (the perceived strength of the odour) of an ethanolic fragrance composition.

19. (Canceled)

20. A method for conveying, enhancing or modifying an odour on human skin, comprising:
applying a composition according to claim 1 to human skin.

21. A composition according to claim 1, wherein a deliquescent substance is added as an agent for
extending the adhesion of fragrances of an ethanolic fragrance composition, in particular of the readily volatile fragrances (top note), to skin (i.e. not (oral) mucosa) and/or hair, in particular to human skin; and/or
increasing or extending the diffusivity (spatial effect) of an ethanolic fragrance composition, in particular of the readily volatile fragrances (top note), on skin (i.e. not (oral) mucosa) and/or hair, in particular on human skin; and/or
conveying or enhancing a pleasant skin sensation of an ethanolic fragrance composition.
